(19) United States
(12) Patent Application Publication
(10) Pub. No.: US 2025/0099119 A1
(43) Pub. Date: Mar. 27, 2025

(54) THROMBECTOMY APPARATUS

(71) Applicant: Boston Scientific Scimed, Inc., Maple Grove, MN (US)

(72) Inventors: Paul F. Chouinard, Maple Grove, MN (US); James Burke Lynch, Minneapolis, MN (US); Katherine Gingras, Rosemount, MN (US); Winnie Tang, Santa Clara, CA (US); Timothy Lawrence Rubesch, Blaine, MN (US)

(73) Assignee: Boston Scientific Scimed, Inc., Maple Grove, MN (US)

(21) Appl. No.: 18/892,627

(22) Filed: Sep. 23, 2024

Related U.S. Application Data
(60) Provisional application No. 63/540,599, filed on Sep. 26, 2023.

(51) Int. Cl. A61B 17/22; A61B 17/00

(52) U.S. Cl. CPC A61B 17/22031; A61B 17/00234; A61B 2017/00238; A61B 2017/00305; A61B 2017/00862; A61B 2017/22034

(57) ABSTRACT

A thrombectomy apparatus for removing a clot from a vessel includes a catheter defining a lumen extending proximally from a distal end. A funnel is secured to the distal end and extends distally therefrom, the funnel includes a braided inner layer and a braided outer layer, the braided outer layer folded over the braided inner layer and adapted to constrain the braided inner layer. A tractor is adapted to extend over an outer surface of the catheter and the funnel in an un-inverted configuration and to extend in an inverted configuration through the funnel and into the lumen, the tractor adapted to invert by rolling over the distal end of the funnel when the tractor moves proximally. A puller extends through the lumen and is secured to an end of the tractor disposed within the lumen.

### CROSS-REFERENCE TO RELATED APPLICATIONS

This application claims the benefit of priority under 35 U.S.C. § 119 of U.S. Provisional Application No. 63/540,599, filed Sep. 26, 2023, the entire disclosure of which is hereby incorporated by reference.

### TECHNICAL FIELD

The present disclosure relates generally to medical devices. More particularly, the present disclosure pertains to medical devices for performing thrombectomy procedures.

### BACKGROUND

A wide variety of intracorporeal medical devices have been developed for medical use, for example, intravascular use. Some of these devices include thrombectomy apparatuses. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for thrombectomy apparatuses. An example may be found in a thrombectomy apparatus for removing a clot from a vessel. The thrombectomy apparatus includes a catheter having a distal region including a distal end, the catheter defining a lumen extending proximally from the distal end, the catheter having an outer surface. A funnel is secured to the distal end and extends distally therefrom, the funnel includes a braided inner layer and a braided outer layer, the braided outer layer folded over the braided inner layer and adapted to constrain the braided inner layer. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen. A puller extends through the lumen and is secured to an end of the tractor disposed within the lumen.

Alternatively or additionally, the tractor may be adapted to engage the clot and pull the clot proximally into the funnel as the tractor inverts over the distal end of the funnel.

Alternatively or additionally, the tractor may include a flexible tube having a plurality of loops that lie flat before reaching the distal end of the catheter and after passing the distal end of the catheter, but temporarily extend radially outwardly as the tractor inverts over the distal end of the catheter, the plurality of loops temporarily extending radially outward as the tractor inverts over the distal end of the catheter engage the clot and pull the clot proximally.

Alternatively or additionally, the braided outer layer may be adapted to constrain against the braided inner layer expanding radially as a result of axial compression as the tractor is pulled over the distal end of the funnel.

Alternatively or additionally, the braided inner layer may have a first braid angle, the braided outer layer may have a second braid angle, and the second braid angle may be greater than the first braid angle.

Alternatively or additionally, the funnel may be formed from a unitary braided member folded over on itself, the braided inner layer including a first portion of the unitary braided member and the braided outer layer including a second portion of the unitary braided member.

Alternatively or additionally, the braided outer layer may include additional filaments.

Alternatively or additionally, the braided outer layer may include some single filaments extending both clockwise and counterclockwise and some double filaments extending both clockwise and counterclockwise.

Alternatively or additionally, the braided outer layer may include about half single filaments extending both clockwise and counterclockwise and about half double filaments extending both clockwise and counterclockwise.

Alternatively or additionally, the braided inner layer may not include any additional filaments.

Alternatively or additionally, the braided outer layer includes a varying PPI count along its length.

Alternatively or additionally, the braided inner layer comprises a constant PPI count along its length.

Another example may be found in a thrombectomy apparatus for removing a clot from a vessel. The thrombectomy apparatus includes a catheter having a distal end and a lumen extending proximally from the distal end, and a funnel extending distally from the distal end. The funnel includes a braided inner layer and a braided outer layer, the braided outer layer folded over the braided inner layer and adapted to constrain the braided inner layer. A tractor is adapted to extend outside the catheter and funnel in an un-inverted configuration and to extend in an inverted configuration into an interior of the funnel, the tractor adapted to invert by rolling over the distal end of the funnel when the tractor is pulled proximally within the lumen.

Alternatively or additionally, the braided inner layer may have a first braid angle, the braided outer layer may have a second braid angle, and the second braid angle may be greater than the first braid angle.

Alternatively or additionally, the funnel may be formed from a braided member folded over on itself, the braided inner layer including a first portion of the braided member and the braided outer layer including a second portion of the braided member.

Alternatively or additionally, the braided outer layer may include additional filaments.

Alternatively or additionally, the braided outer layer may include some single filaments extending both clockwise and counterclockwise and some double filaments extending both clockwise and counterclockwise.

Alternatively or additionally, the braided outer layer may include about half single filaments extending both clockwise and counterclockwise and about half double filaments extending both clockwise and counterclockwise.

Alternatively or additionally, the braided outer layer includes a varying PPI count along its length.

Another example may be found in a thrombectomy apparatus for removing a clot from a vessel. The thrombectomy apparatus includes a catheter having a distal end and a lumen extending proximally from the distal end. A funnel extends distally from the distal end. The funnel includes a braided inner layer having a first braid angle and a braided outer layer having a second braid angle, the braided outer layer folded over the braided inner layer, the second braid angle greater than the first braided angle. A tractor is adapted to extend outside the catheter and funnel in an un-inverted configuration and to extend in an inverted configuration into an interior of the funnel, the tractor adapted to invert by rolling over the distal end of the funnel when the tractor is pulled proximally within the lumen.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative thrombectomy apparatus shown in a starting position proximate a clot within a blood vessel;

FIG. 2 is a schematic view of the illustrative thrombectomy apparatus of FIG. 1, shown in a finishing position after having captured the clot from within the blood vessel;

FIG. 3 is a schematic view of an illustrative tractor disposed over an illustrative funnel as part of the illustrative thrombectomy apparatus of FIG. 1;

FIG. 4 is a schematic view of an illustrative braiding pattern;

FIG. 5 is a schematic view of a braided member resulting from the illustrative braiding pattern of FIG. 4;

FIG. 6 is a schematic view of an illustrative funnel formed from the illustrative braiding pattern of FIG. 4;

FIG. 7 is a schematic view of an illustrative outer braided layer of a funnel formed from a braiding pattern in which the outer braided layer maintains a constant braid angle from proximal end to distal end of the outer braided layer;

FIG. 8 is a schematic view of an illustrative braiding pattern with extra filaments;

FIGS. 9 and 10 are schematic views of an illustrative funnel undergoing compressive testing; and

FIG. 11 is a graphical representation of experimental results from illustrative funnels undergoing compressive testing.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

Thrombectomy is a process whereby a thrombus, or clot, is removed from within a blood vessel. FIG. 1 is a schematic view of an illustrative thrombectomy apparatus 10 that is shown positioned within an example blood vessel 12. A thrombus or clot 14 is schematically shown within the blood vessel 12. The thrombectomy apparatus 10 includes a catheter 16 that extends from a proximal region 18 to a distal region 20 and includes a distal end 22. A lumen 24 extends through the catheter 16. The catheter 16 includes an outer surface 26. A funnel 28 is secured to the distal end 22 and extends distally from the distal end 22. In some instances, the funnel 28 is a braided structure including a braided inner layer and a braided outer layer, as will be shown in subsequent drawings. As will be discussed, in some instances the braided outer layer may be adapted to constrain the braided inner layer from expanding too far in a radially outward direction as a result of an axial compressive force.

A tractor 30 is adapted to extend over the outer surface 26 of the catheter 16 and the funnel 28 in an un-inverted configuration and to extend in an inverted configuration through the funnel 28 and into the lumen 24. In some instances, the tractor 30 may be adapted to invert by rolling over a distal end 32 of the funnel 28 when the tractor 30 moves proximally through the lumen 24. It will be appreciated that a portion of the tractor 30 within the lumen 24, the inverted portion, moves proximally while another portion of the tractor 30, outside of the catheter 16, move distally. The thrombectomy apparatus 10 includes an elongate member 34 that extends through the lumen 24 and is operably coupled with an end 36 of the tractor 30 that is disposed within the lumen 24. In some instances, the tractor 30 is adapted to engage the clot 14 and to pull the clot 14 proximally into the funnel 28 and thus into the lumen 24 as the tractor 30 inverts over the distal end 32 of the funnel 28.

The thrombectomy apparatus 10 includes an elongate member 34 that is adapted to extend through the lumen 24 within the catheter 16. The elongate member 34 is secured to the end 36 of the tractor 30 that is disposed within the lumen 24. In some instances, the elongate member 34 may be a catheter. In some instances, the elongate member 34 may be a pull wire or other pull member that is flexible enough to bend within the lumen 24 as the catheter 16 navigates the vasculature, but strong enough to allow a user to pull the elongate member 34 proximally in order to pull the tractor 30 proximally into the funnel 28 and thus into the lumen 24. In some instances, the elongate member 34 may be adhesively secured to the tractor 30. In some instances, the elongate member 34 may be welded to the tractor 30.

In some instances, the thrombectomy apparatus 10 may include a sheath 38. The sheath 38 may overly the catheter 16 and the tractor 30. In some instances, the sheath 38 helps prevent the tractor 30 from engaging or otherwise contacting the walls of the blood vessel 12. In some instances, the sheath 38 may have an inner diameter sufficient to prevent the tractor 30 from engaging the walls of the blood vessel 12 while still allowing the tractor 30 to move relative to the outer surface 26 of the catheter 16 as the tractor 30 moves. In some instances, the sheath 38 or at least an inner layer or coating may be formed of a lubricious material such as but not limited to a fluoropolymer. As an example, the sheath 38 may be formed of, or may at least have an inner layer or coating that is formed of a fluoropolymer such as polytetrafluoroethylene (PTFE), commonly known as Teflon®.

In some instances, the sheath 38 may be positioned further distally relative to the funnel 28 during advancement of the thrombectomy apparatus 10 through the vasculature. In some instances, the sheath 38 may constrain the funnel 28 into a smaller diameter configuration while the thrombectomy apparatus 10 is advanced through the vasculature. Once a desired location has been reached, the sheath 38 may be withdrawn proximally a short distance in order to allow the funnel 28 to expand into a conical shape, as shown. Prior to subsequent removal of the thrombectomy apparatus 10, the sheath 38 may be advanced distally a short distance in order to once again collapse the funnel 28 into a smaller diameter configuration for navigation.

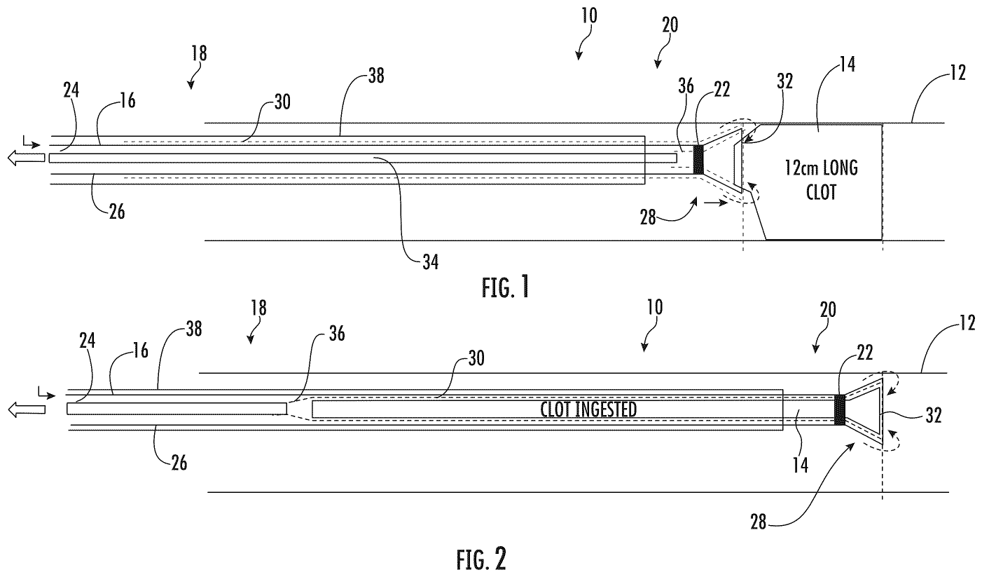

As noted, FIG. 1 shows the thrombectomy apparatus 10 positioned within the blood vessel 12, with the distal end 32 of the funnel 28 positioned adjacent or even in contact with the clot 14. By withdrawing the elongate member 34 proximally, the tractor 30 is caused to move over the distal end 32 of the funnel 28, and to pass through an interior of the funnel 28 and into the lumen 24. In some instances, as the tractor 30 moves from an un-inverted configuration proximal of the distal end 32 of the funnel 28, to an inverted configuration as the tractor 30 moves beyond the distal end 32 of the funnel 28, the tractor 30 is adapted to grab the clot 14 and to pull the clot 14 into the funnel 28 and into the lumen 24. The culmination of this may be seen in FIG. 2, which shows the thrombectomy apparatus 10 in a finishing position in which the clot 14 has been completely pulled into an interior of the thrombectomy apparatus 10. As can be seen in FIG. 2, the clot 14 is positioned within the lumen 24 and within the funnel 28. At this point, withdrawal of the thrombectomy apparatus 10 means that clot 14 will have been removed from the patient.

It will be appreciated that the clot 14 has a length when disposed within the blood vessel 12. In some instances, during the process of capturing the clot 14, the thrombectomy apparatus 10 will move distally. In some instances, the thrombectomy apparatus 10 will move distally a distance about equal to the length of the clot 14 when the clot 14 is still in the blood vessel 12. If the undisturbed clot 14 has a length of 12 centimeters, the thrombectomy apparatus 10 may move distally a distance of about 12 centimeters in the process of capturing the clot 14. In some instances, depending on the nature of the clot 14, the thrombectomy apparatus 10 may move distally a distance greater than the length of the undisturbed clot 14, for example.

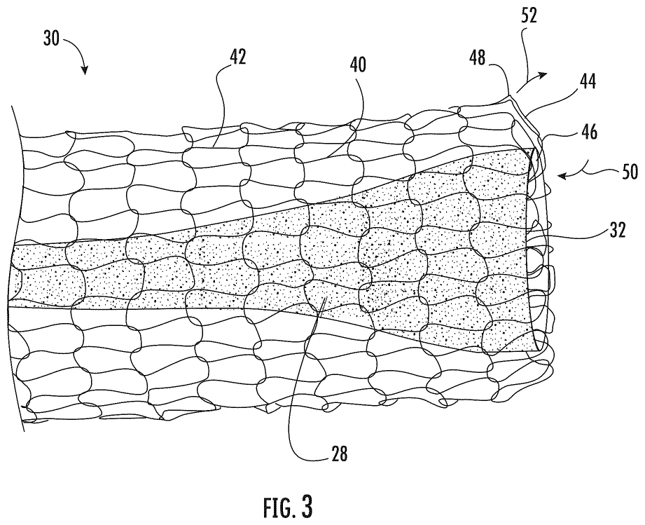

FIG. 3 is a schematic view of the tractor 30 disposed over the funnel 28. In some instances, as shown, the tractor 30 may be formed from a plurality of loops 40. In some instances, the tractor 30 may be formed by knitting one or more filaments 42 into the plurality of loops 40. A variety of different knitting patterns may be utilized, for example. It will be appreciated that the portion of the tractor 30 shown outside of the funnel 28 is in an un-inverted configuration while the portion of the tractor 30 inside the funnel 28 (not visible) is in an inverted configuration. The tractor 30 inverts from the un-inverted configuration to the inverted configuration as the tractor 30 moves around the distal end 32 of the funnel 28. With particular attention to a specific loop 44, it can be seen that the loop 44 has a leading edge 46 and a trailing edge 48. As the loop 44 pivots around the distal end 32 of the funnel 28, the leading edge 46 moves in a direction indicated by an arrow 50 while the trailing edge 46 moves in a direction indicated by an arrow 52. Accordingly, as each of the loops 44 pivot around the distal end 32 of the funnel 28, each loop 44 will temporarily extend radially outwardly as the loop 44 pivots. Otherwise, each of the loops 44 will lay flat, particularly as tension is placed on the tractor 30. Additional details regarding the thrombectomy apparatus 10, including the catheter 16, the tractor 30 and the elongate member 34, may be found in U.S. Pat. Nos. 9,962,178; 10,835,269; US 2022/0257269 and US 2022/0287053, each of which are incorporated by reference herein.

It will be appreciated that as the tractor 30 is pulled into the funnel 28 and into the lumen 24 by withdrawing the elongate member 34 proximally, and as a result the tractor 30 inverts over the distal end 32 of the funnel 28, that an axial compressive force is applied to the funnel 28 by the tractor 30. In some instances, the funnel 28 may be adapted to resist or otherwise accommodate this axial compressive force such that the funnel 28 is able to retain its tapered shape. In some instances, as will be discussed, the funnel 28 may be a braided structure. Braided structures can have a tendency to increase in diameter in response to a decrease in length, and of course the inverse is true as well. A braided structure can have a tendency to decrease in diameter in response to an increase in length. In some instances, the funnel 28 may be a braided structure having an inner braided layer and an outer braided layer. In some instances, the outer braided layer may be adapted to constrain the inner braided layer from radially expanding too far in response to an applied axial force. The resulting composite structure, including the inner braided layer and the outer braided layer, may be considered as a conflicting braid funnel.

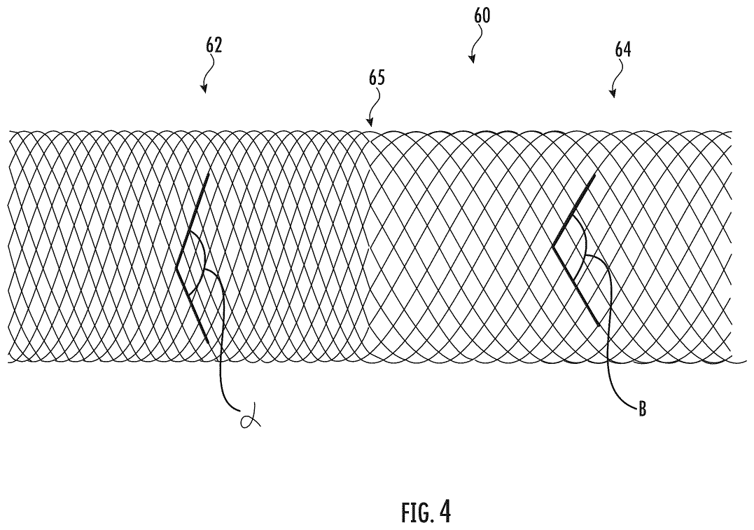

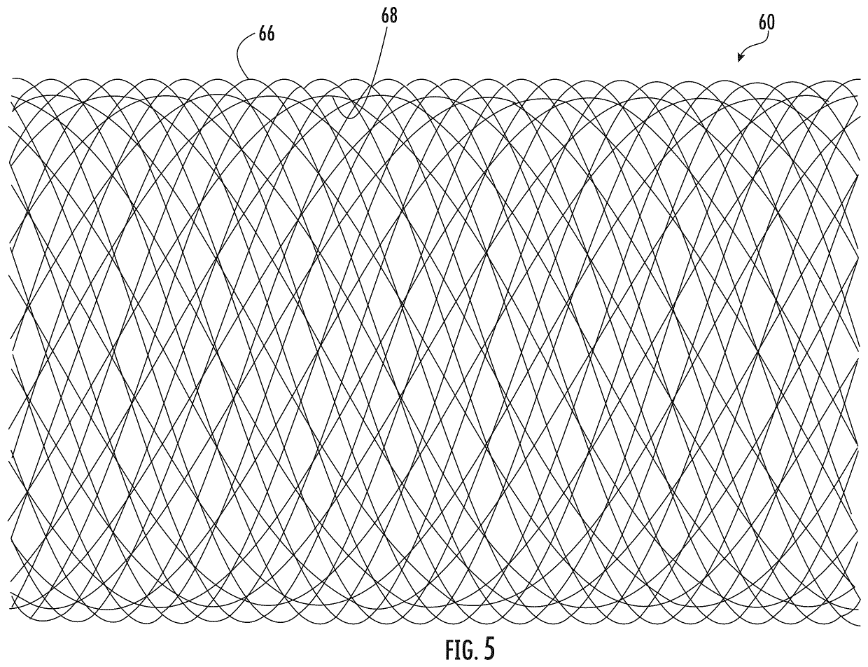

FIG. 4 is a schematic view of an illustrative braided member 60 that may be used in forming the funnel 28. It will be appreciated that the braided member 60 is illustrated two-dimensionally, even though the braided member 60 is actually a three dimensional construct. The braided member 60 may be considered as including a first portion 62 that will ultimately form an outer braid layer and a second portion 64 that will ultimately form an inner braid layer. The braided member 60 may be considered as being a unitary or continuous braid, with the individual filament or filaments forming the braided member 60 extending continuously through both the first portion 62 and the second portion 64. In some instances, the braided member 60 will undergo a change in braid angle at a fold location 65. In some instances, the braided member 60 may additionally or alternatively undergo a change in diameter at the fold location 65. For example, the second portion 64 may be formed to have a smaller diameter than the first portion 62, which may facilitate forming a dual layer braided structure as the second portion 64 ultimately is manipulated into an interior of the first portion 62.

As shown, the first portion 62 has a braid angle indicated as a (alpha) and the second portion 64 has a braid angle indicated as β (beta). As can be seen, the braid angle α (alpha) is larger than the braid angle β (beta). In some instances, the braid angle α (alpha) may be at least five percent or more greater than the braid angle β (beta). In some instances, the braid angle α (alpha) may be at least ten percent or more greater than the braid angle β (beta). In some instances, the braid angle α (alpha) may be at least fifteen percent or more greater than the braid angle β (beta). In some instances, the braid angle α (alpha) may be at least twenty percent or more greater than the braid angle β (beta). In some instances, the braid angle α (alpha) may be in a range of 90 to 150 degrees while the braid angle β (beta) may be in a range of 90 to 140 degrees, with the caveat that the braid angle α (alpha) is larger than the braid angle β (beta). As an example, the braid angle α (alpha) may be about 135 to 145 degrees while the braid angle β (beta) may be about 115 degrees to about 125 degrees. Because the first portion 62 has a higher braid angle, the first portion will not radially expand in response to an applied axial force as much as the second portion 64 will. Because the first portion 62 will form the outer braided layer, the first portion 62 can be seen as constraining the second portion 64 (which will form the inner braided layer) from expanding as much as the second portion 64 would otherwise expand in response to an applied axial force.

In order to form the funnel 28, the braided member 60 may be manipulated such that the second portion 64 is disposed inside of the first portion 62. In some instances, this may include pushing the second portion 64 inside of the first portion 62. In some instances, this may include folding the first portion 62 over on top of the second portion 64. As noted, the first portion 62 will form the outer braided layer and the second portion 64 will form the inner braided layer. This may be seen in FIG. 5, which shows the braided member 60 with an outer braided layer 66 that corresponds to the first portion 62 and an inner braided layer 68 that corresponds to the second portion 64.

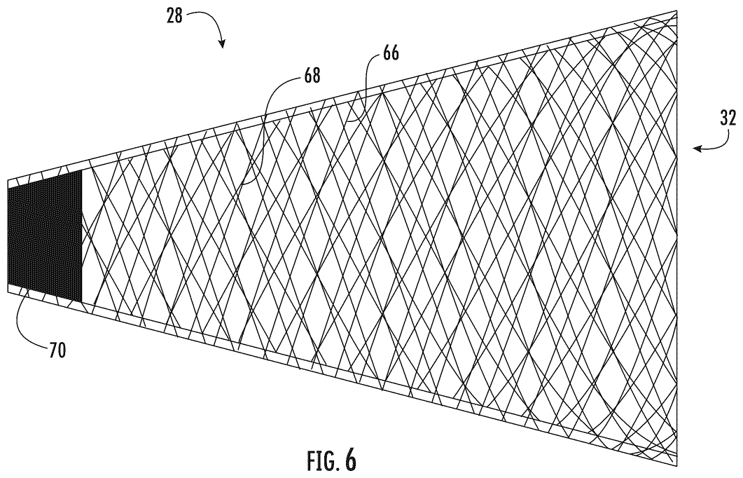

FIG. 6 shows the funnel 28 in finished form. The funnel 28 includes a cuff 70 that holds the inner braided layer 68 in a conical shape. The cuff 70 may be a polymeric sleeve, for example. In some instances, the braided member 60 as shown in FIG. 5 may be compressed into a conical shape in forming the funnel 28. To an extent, FIG. 6 may be considered as being schematic in nature because FIG. 6 does not show the compression that will occur in the smaller diameter portions of the funnel 28 as the braided member 60 (FIG. 4) is compressed into the ultimate conical shape of the funnel 28. The outer braided layer 66 will constrain the inner braided layer 68 from radially expanding too far as a result of the applied axial force from the tractor 30 being pulled into the funnel 28.

In some instances, compressing the braided member 60 down into the funnel 28 results in the braid angles being reduced in the smaller diameter portions of the funnel 28. As an example, the funnel 28 may taper from an maximum outer diameter measured at the mouth of the funnel 28 of about 10 millimeters to a minimum outer diameter measured at the end opposing the mouth of the funnel 28 of about 4 millimeters. If left unconstrained, the 4 millimeter diameter portion of the braid could expand out to about 10 millimeters. In some instances, the braided member 60 may be modified such the braided member 60 achieves the desired conical shape when undergoing an axial tensile force.

In some instances, the braiding pattern used to form the braided member 60 may provide a varying PPI in order to limit the maximum expansion diameter of the braided member 60. In some instances, at least the outer braided layer may be modified in this way. The inner braided layer may be, but even if the inner braided layer is not modified in this way, the outer braided layer that is modified to form the desired conical funnel shape under compression will limit the radial expansion of the inner braided layer. This may be achieved, for example, by braiding on a shaped mandrel with a single PPI count programmed. This may be achieved, for example, by braiding on a tubular mandrel with a varying PPI count.

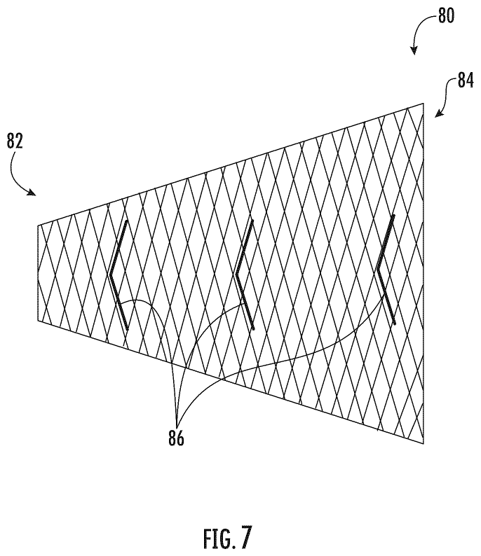

FIG. 7 is a schematic view of an illustrative outer braided layer 80 formed from a braiding pattern having a varying PPI such that the outer braided layer 80 maintains a constant braid angle from a proximal end 82 to a distal end 84 of the outer braided layer 80 when compressed. In particular, the outer braided layer 80 has a constant large braid angle, as indicated at 86. The large braid angle 86 indicates that the outer braid layer 80 has been compressed to its limit, meaning that the outer braid layer 80 is at its maximum radial expansion diameter. As a result, the outer braid layer 80 is able to constrain an inner braid layer (not shown) from radially expanding too far.

In some instances, the funnel 28 may be formed from a braided outer layer that includes additional filaments. For example, the funnel 28 may be formed from a braided outer layer in which some of the windings are formed from single filaments while others of the windings are formed from double or triple filaments, for example. The braided outer layer may include some single filaments that extend both clockwise and counterclockwise and some double filaments extending both clockwise and counterclockwise. The braided outer layer may include about half single filaments extending both clockwise and counterclockwise and about half double filaments extending both clockwise and counterclockwise. In some instances, adding additional filaments to the braided outer layer can help the braided outer layer resist radial expansion as a result of being compressed, and thus can help constrain an inner braided layer of the funnel 28.

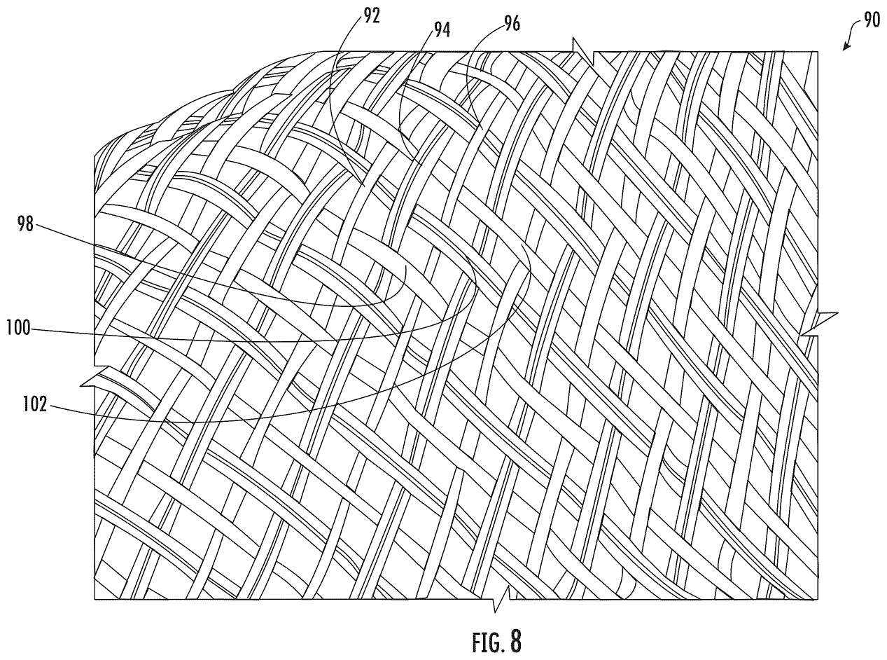

FIG. 8 is a schematic illustration of a braiding pattern 90 that may be used in forming a braided outer layer of the funnel 28. The same braiding pattern 90 may be utilized in forming an braided inner layer of the funnel 28. In some instances, the braiding pattern 90 may be used in forming a braided outer layer of the funnel 28 while another braiding pattern (not shown) may be used in forming a braided inner layer of the funnel 28. In the braiding pattern 90, half of the windings in the clockwise direction are single filaments and half of the windings in the counterclockwise direction are single filaments. Half of the windings in the clockwise direction are double filaments and half of the windings in the counterclockwise direction are double filaments. To illustrate, in a first direction (as clockwise and counterclockwise are dependent upon viewing position) a winding 92 is a single filament, a winding 94 is a double filament and a winding 96 is a single filament. In an opposing direction, a winding 98 is a single filament, a winding 100 is a double filament, and a winding 102 is a single filament.

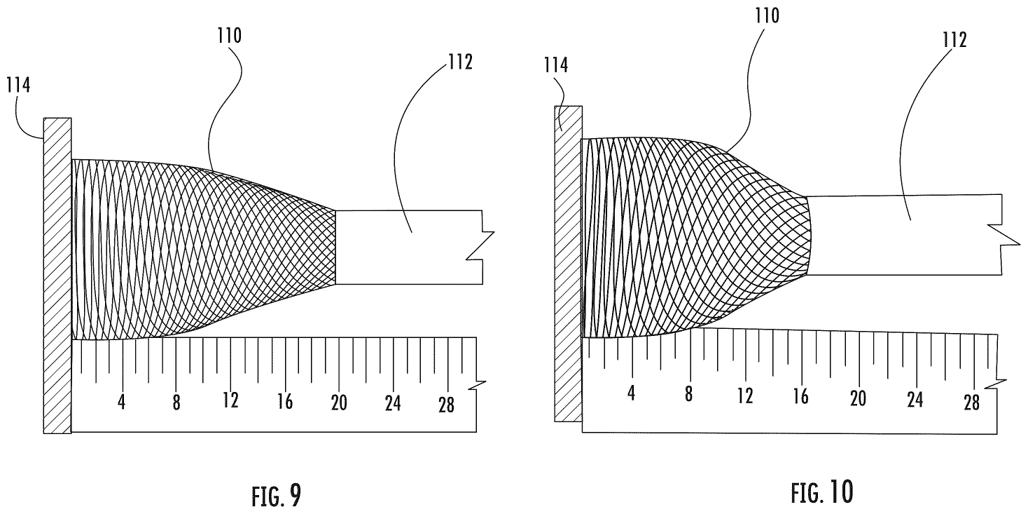

FIGS. 9 and 10 are schematic views of an illustrative funnel 110 undergoing compressive testing. The funnel 110 includes 50 percent additional filaments in both the braided inner layer and the braided outer layer of the funnel 110. The funnel 110 is secured to a shaft 112, and can be seen as being compressed against a rigid surface 114. In FIG. 9, the funnel 110 is in contact with the rigid surface 114, but is not yet under compression. In FIG. 10, the funnel 110 is under compression. This can be seen by noting that in FIG. 9, the funnel 110 has an overall length of about 20 millimeters, while in FIG. 10 the funnel 110 has an overall length of about 16 millimeters. It can be seen by comparing FIG. 9 and FIG. 10 that while the funnel 110 has shortened axially and increased in diameter radially, the funnel 110 has retained its conical shape.

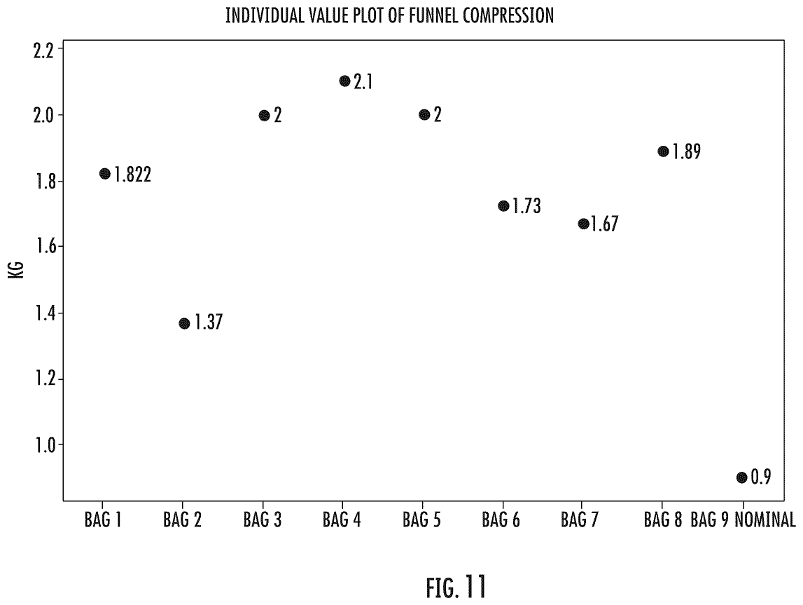

FIG. 11 is a graphical representation of experimental results from illustrative funnels undergoing compressive testing. The results compare a control, labeled as nominal, with a total of 9 other funnels that were tested to maximum failure. The control did not have any extra filaments, while the other tested funnels each had 50 percent double filaments. The control failed at a compression of 0.9 kilograms while the inventive funnels failed at compression values ranging from 1.37 kilograms to 2.1 kilograms. As can be seen, using 50 percent double filaments provides a considerable benefit to how well the funnel withstands compressive forces.

The materials that can be used for the various components of the thrombectomy apparatus, and the various elements thereof disclosed herein may include those commonly associated with medical devices. For simplicity purposes, the following discussion refers to the thrombectomy apparatus. However, this is not intended to limit the devices and methods described herein, as the discussion may be applied to other elements, members, components, or devices disclosed herein. In some instances, the thrombectomy apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A thrombectomy apparatus for removing a clot from a vessel, the thrombectomy apparatus comprising:
a catheter having a distal region including a distal end, the catheter defining a lumen extending proximally from the distal end, the catheter having an outer surface;
a funnel secured to the distal end and extending distally therefrom, the funnel including:
a braided inner layer; and
a braided outer layer, the braided outer layer folded over the braided inner layer, the braided outer layer adapted to constrain the braided inner layer;
a tractor adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen;
an elongate member extending through the lumen and secured to an end of the tractor disposed within the lumen.

2. The thrombectomy apparatus of claim 1, wherein the tractor is adapted to engage the clot and pull the clot proximally into the funnel as the tractor inverts over the distal end of the funnel.

3. The thrombectomy apparatus of claim 2, wherein the tractor comprises a flexible tube comprising a plurality of loops that lie flat before reaching the distal end of the catheter and after passing the distal end of the catheter, but temporarily extend radially outwardly as the tractor inverts over the distal end of the catheter, the plurality of loops temporarily extending radially outward as the tractor inverts over the distal end of the catheter engage the clot and pull the clot proximally.

4. The thrombectomy apparatus of claim 1, wherein the braided outer layer is adapted to constrain against the braided inner layer expanding radially as a result of axial compression as the tractor is pulled over the distal end of the funnel.

5. The thrombectomy apparatus of claim 1, wherein:
the braided inner layer has a first braid angle;
the braided outer layer has a second braid angle; and
the second braid angle is greater than the first braid angle.

6. The thrombectomy apparatus of claim 5, wherein the funnel is formed from a unitary braided member folded over on itself, the braided inner layer comprising a first portion of the unitary braided member and the braided outer layer comprises a second portion of the unitary braided member.

7. The thrombectomy apparatus of claim 1, wherein the braided outer layer comprises additional filaments.

8. The thrombectomy apparatus of claim 7, wherein the braided outer layer comprises some single filaments extending both clockwise and counterclockwise and some double filaments extending both clockwise and counterclockwise.

9. The thrombectomy apparatus of claim 8, wherein the braided outer layer comprises about half single filaments extending both clockwise and counterclockwise and about half double filaments extending both clockwise and counterclockwise.

10. The thrombectomy apparatus of claim 7, wherein the braided inner layer does not comprise any additional filaments.

11. The thrombectomy apparatus of claim 1, wherein the braided outer layer includes a varying PPI count along its length.

12. The thrombectomy apparatus of claim 11, wherein the braided inner layer comprises a constant PPI count along its length.

13. A thrombectomy apparatus for removing a clot from a vessel, the thrombectomy apparatus comprising:
a catheter having a distal end and a lumen extending proximally from the distal end;
a funnel extending distally from the distal end, the funnel including:
a braided inner layer; and
a braided outer layer, the braided outer layer folded over the braided inner layer, the braided outer layer adapted to constrain the braided inner layer; and
a tractor adapted to extend outside the catheter and funnel in an un-inverted configuration and to extend in an inverted configuration into an interior of the funnel, the tractor adapted to invert by rolling over the distal end of the funnel when the tractor is pulled proximally within the lumen.

14. The thrombectomy apparatus of claim 13, wherein:
the braided inner layer has a first braid angle;
the braided outer layer has a second braid angle; and
the second braid angle is greater than the first braid angle.

15. The thrombectomy apparatus of claim 14, wherein the funnel is formed from a braided member folded over on itself, the braided inner layer comprising a first portion of the braided member and the braided outer layer comprises a second portion of the braided member.

16. The thrombectomy apparatus of claim 13, wherein the braided outer layer comprises additional filaments.

17. The thrombectomy apparatus of claim 16, wherein the braided outer layer comprises some single filaments extending both clockwise and counterclockwise and some double filaments extending both clockwise and counterclockwise.

18. The thrombectomy apparatus of claim 17, wherein the braided outer layer comprises about half single filaments extending both clockwise and counterclockwise and about half double filaments extending both clockwise and counterclockwise.

19. The thrombectomy apparatus of claim 13, wherein the braided outer layer includes a varying PPI count along its length.

20. A thrombectomy apparatus for removing a clot from a vessel, the thrombectomy apparatus comprising:
a catheter having a distal end and a lumen extending proximally from the distal end;
a funnel extending distally from the distal end, the funnel including:
a braided inner layer having a first braid angle; and
a braided outer layer having a second braid angle, the braided outer layer folded over the braided inner layer, the second braid angle greater than the first braided angle; and
a tractor adapted to extend outside the catheter and funnel in an un-inverted configuration and to extend in an inverted configuration into an interior of the funnel, the tractor adapted to invert by rolling over the distal end of the funnel when the tractor is pulled proximally within the lumen.
